(19) United States
(12) Patent Application Publication
(10) Pub. No.: US 2004/0110947 A1
(43) Pub. Date: Jun. 10, 2004

(54) Aromatic liver X-receptor modulators

(75) Inventors: Nizal S. Chandrakumar, Vernon Hills, IL (US); Christopher R. Dalton, Mundelein, IL (US); James W. Malecha, Libertyville, IL (US); Michael B. Tollefson, Dardenne Prairie, MO (US); Jennifer Ann Van Camp, Glencoe, IL (US); Philip B. Cox, Grayslake, IL (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/664,229

(22) Filed: Sep. 17, 2003

Related U.S. Application Data
(60) Provisional application No. 60/411,362, filed on Sep. 17, 2002.
(60) Provisional application No. 60/436,240, filed on Dec. 23, 2002.

(51) Int. Cl. C07D 417/02; C07D 413/02; C 07D 4 3/02

(52) U.S. Cl. 544/60; 544/124; 544/295; 544/360; 546/268.1

(57) ABSTRACT

The present invention is directed to selective LXR modulators, small molecule compounds corresponding to Formula I and is further directed to a process of treating a condition in a mammal that is modulated by LXR using a therapeutically effective dose of a compound of Formula I.

### REFERENCE TO RELATED APPLICATIONS

This application is a non-provisional application claiming priority from provisional applications Serial No. 60/411,362 filed Sep. 17, 2002 and Serial No. 60/436,240 filed Dec. 23, 2002, the entire contents of which are hereby incorporated herein by reference in its entirety.

### BACKGROUND

Liver X-receptors (LXRs) are nuclear receptors that regulate the metabolism of several important lipids, including cholesterol and bile acids. Most of the cholesterol in plasma is transported on three major lipoprotein classes; VLDL cholesterol (VLDL-C), LDL cholesterol (LDL-C) and HDL cholesterol (HDL-C). Total cholesterol is the sum of all three lipoproteins. Both VLDL-C and LDL-C are associated with atherogenic processes while HDL-C is believed to facilitate cholesterol removal from tissues (e.g. atherosclerotic plaques) and thus have a protective effect on coronary heart disease.

LXR represents a novel intervention point to regulate the reverse cholesterol transport (RCT) pathway, i.e., the removal of cholesterol from peripheral tissues/cells and subsequent uptake via the liver for disposal. Removal of cellular cholesterol requires active transport of free cholesterol across the plasma membrane and onto HDL particles. This transfer of cholesterol from inside the cell and onto HDL in the plasma is mediated by ATP binding cassette 1 (ABCA1) transporter protein. The observation that LXR is a key transcriptional activator of ABCA1 in the macrophage, suggests that induction of LXR will lead to an increase in cholesterol efflux from the macrophage. In addition, it is known that LXR regulates the induction of other genes involved in RCT such as apoE and cholesterol ester transport protein (CETP), suggesting that activating the LXR pathway should also lead to increased uptake of cholesterol by the liver. Thus, activation of LXR by a small molecule ligand will lead to an up-regulation of ABCA1 and induction of the reverse cholesterol transport pathway thereby increasing cholesterol efflux to HDL-C and reducing the cholesterol content of atherosclerotic plaques.

### SUMMARY OF THE INVENTION

In general, the present invention is directed to selective LXR modulators, small molecule compounds corresponding to Formula I and the isomers, tautomers, salts and prodrugs thereof:

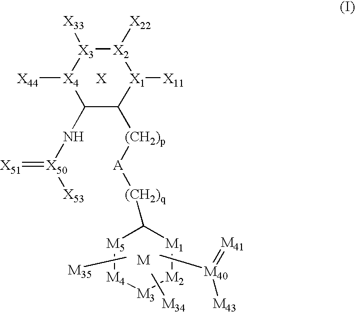

wherein:

the X ring and the M ring are independently aromatic rings;

A is oxygen, sulfur, sulfoxide, sulfone, —NHC(═A₂)— or —C(═A₂)NH—;

A₂ is oxygen or sulfur;

M₁, M₂, M₃, M₄, and M₅ are independently a bond, carbon, nitrogen, oxygen or sulfur, provided, however, no more than one of M₁, M₂, M₃, M₄, and M₅ is a bond;

M₃₄ and M₃₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl, or M₃₄ and M₃₅ are bonded to adjacent carbon atoms and together with the atoms to which they are bonded form a fused ring system;

M₄₀ is carbon, sulfur or sulfoxide;

M₄₁ is oxygen, sulfur, or NM₄₂;

M₄₂ is hydrogen, hydrocarbyl, or substituted hydrocarbyl; and

M₄₃ is hydrogen, hydrocarbyl, substituted hydrocarbyl, hydrocarbyloxy, substituted hydrocarbyloxy, amino, hydrocarbylthio, or substituted hydrocarbylthio;

p and q are independently 0, 1,or 2;

X₁, X₂, X₃, and X₄ are independently a bond, carbon, nitrogen, oxygen or sulfur, provided, however, no more than one of X₁, X₂, X₃, and X₄ is a bond;

X₁₁, X₂₂, X₃₃, and X_{44,} are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl; provided, however, X₁₁, X₂₂, X₃₃, or X₄₄ is not present when X₁, X₂, X₃ or X₄, respectively, is a bond;

X₅₀ is carbon, sulfur or sulfoxide,

X₅₁ is oxygen, sulfur, or NX₅₂,

X₅₂ is hydrogen, hydrocarbyl, or substituted hydrocarbyl; and

X₅₃ is hydrogen, hydrocarbyl, substituted hydrocarbyl, heterocyclo, or amino.

The present invention is further directed to a process of treating a condition in a mammal that is modulated by LXR. The process comprises administering to a mammal in need thereof a therapeutically effective dose of a compound of Formula I.

Other aspects of the invention will be in part apparent and in part pointed out hereinafter.

### DETAILED DESCRIPTION OF THE PREFERRED EMBODIMENTS

In general, the present invention is directed to small molecule compounds corresponding to Formula I and the isomers, tautomers, salts and prodrugs thereof and their use as LXR modulators.

In one embodiment, the X ring and the M ring of Formula I are independently a six membered aromatic ring such as a benzene, pyridine or pyrimidine ring, or a 5-membered heteroaromatic ring such as a furan, thiophene, oxazole, pyrazole, pyrrole, thiazole, imidazole or isoxazole ring. For example, the X ring may be a 5-membered ring and the M ring may be a 6-membered ring, or vice versa.

As depicted in Formula I, the bridge between the X and the M rings is —(CH₂)ₚ—A—(CH₂)_{q}— wherein p, q, and A are as defined in connection with Formula I. In one embodiment, the sum of p and q does not exceed 2. In another embodiment, the sum of p and q is 1; for example, p may be 0 when q is 1. In each of these separate embodiments, A may be sulfur, sulfoxide, sulfone, —NHC(═A₂)— or —C(═A₂)NH— wherein A₂ is oxygen or sulfur. In one particular embodiment, the sum of p and q is 1 and A is sulfur. For example, in this particular embodiment, the LXR modulator may correspond to Formula IIA or Formula IIB:

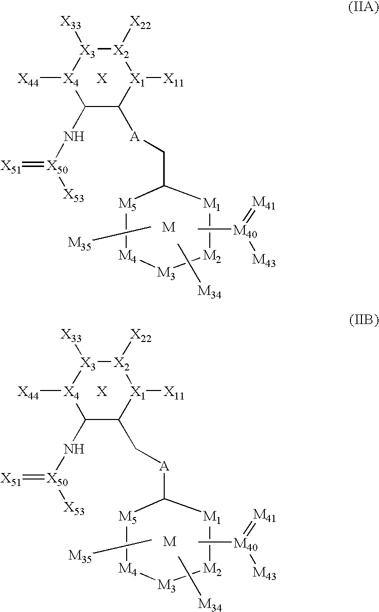

wherein the X ring and the M ring are independently aromatic rings and M₁, M₂, M₃, M₄, M₅, M₃₄, M₃₅, M₄₀, M₄₁, M₄₃, X₁, X₂, X₃, X₄, X₁₁, X₂₂, X₃₃, X₄₄, X₅₀, X₅₁, X₅₂, and X₅₃ are as defined in connection with Formula I.

In a further embodiment, the LXR modulators correspond to Formula IIA or IIB wherein the X ring and the M ring are independently benzene rings. In this embodiment, for example, the compounds correspond to Formula IIIA or IIIB:

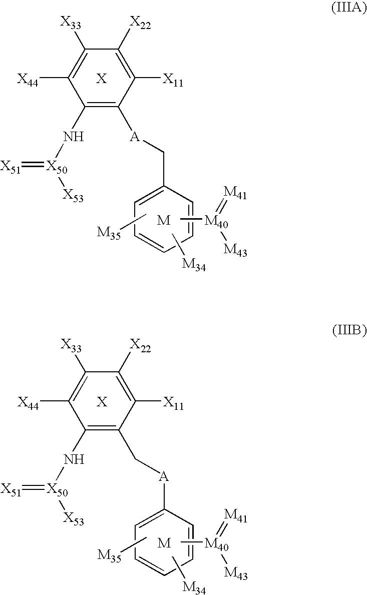

wherein A, X₁₁, X₂₂, X₃₃, X₄₄, X₅₀, X₅₁, X₅₃, M₃₄, M₃₅ M₄₀, M₄₁ and M₄₃ are as defined in connection with Formula I. In one embodiment in which the LXR modulators correspond to Formula IIIA or IIIB, X₅₀ is carbon and X₅₁ is oxygen. In another embodiment in which the LXR modulators correspond to Formula IIIA or IIIB, X₅₃ is heterocyclo, optionally substituted alkyl, or optionally substituted phenyl. In a further embodiment in which the compounds correspond to Formula IIIA or IIIB, X₅₀ is carbon, X₅₁ is oxygen and X₅₃ is heterocyclo, optionally substituted alkyl, or optionally substituted phenyl. For example, in each of these separate embodiments, X₅₃ may be heterocyclo (such as thienyl, pyridyl, piperidinyl, piperazinyl, or 2-oxabicyclo[2.2.1]heptane), linear or branched alkyl (such as methyl, t-butyl, isopropyl, or isobutyl), substituted alkyl (such as trichloromethyl, trifluoromethyl, (CH₂Cl)(CH₃)₂C—, (CH₃C(O)OCH₂)(CH₃)₂C—, or (CH₂OH)(CH₃)₂C—, cycloalkyl (such as cyclohexyl, cyclopentyl, adamantyl, or methylcyclohexane), phenyl, or substituted phenyl (such as 3-chlorophenyl or methoxyphenyl). In addition, in each of the embodiments in which the compounds correspond to Formula IIIA or IIIB, one of X₁₁, X₂₂, X₃₃, and X₄₄ may optionally be hydrogen, alkyl (such as methyl), nitro, or halo (such as chloro or fluoro) while the remainder of X₁₁, X₂₂, X₃₃, X₄₄ are hydrogen. In addition, in each of the embodiments in which the LXR modulators correspond to Formula IIIA or IIIB, M₃₄ and M₃₅ may optionally and independently be selected from hydrogen, alkoxy (such as methoxy), optionally substituted alkyl, or they may be attached to adjacent carbon atoms and, in combination with the carbon atoms to which they are attached, form a fused ring.

In a further embodiment, the LXR modulators correspond to Formula IV:

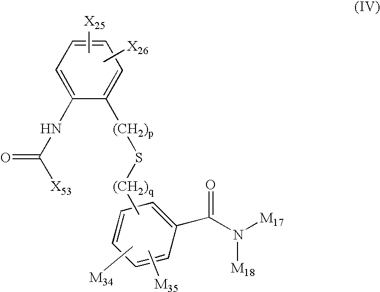

wherein:

p and q are independently 0, 1, or 2;

M₁₇ is hydrogen, hydrocarbyl, substituted hydrocarbyl, hydrocarbyloxy, heterocyclo, amino, or acyl;

M₁₈ is hydrogen, hydrocarbyl, substituted hydrocarbyl, or heterocyclo;

M₃₄ and M₃₅ are independently hydrogen, hydrocarbyl, substituted hydrocarbyl, amino, alkoxy, halogen, or nitro;

X₂₅ and X₂₆ are independently hydrogen, optionally substituted alkyl, nitro or halo, and

X₅₃ is hydrocarbyl, substituted hydrocarbyl or heterocyclo.

In one embodiment in which the LXR modulators correspond to Formula IV, the sum of p and q is one. In another embodiment in which the LXR modulators correspond to Formula IV, p is zero and q is one. In a further embodiment in which the LXR modulators correspond to Formula IV, p is one and q is zero. In each of these separate embodiments in which the LXR modulators correspond to Formula IV, X₂₅, X₂₆, X₅₃, M₁₇, M₁₈, M₃₄ and M₃₅ are as defined in connection with Formula IV. For example, in each of these separate embodiments in which the compounds correspond to Formula IV, X₅₃ may be heterocyclo such as thienyl, pyridyl, piperidinyl, piperazinyl, or 2-oxabicyclo-[2.2.1]heptane, linear or branched alkyl such as methyl, t-butyl, isopropyl, or isobutyl, substituted alkyl such as trichloromethyl, trifluoromethyl, (CH₂Cl)(CH₃)₂C—, (CH₃C(O)OCH₂)(CH₃)₂C—, or (CH₂OH)(CH₃)₂C—, cycloalkyl such as cyclohexyl, cyclopentyl, adamantyl, or methylcyclohexane, phenyl, or substituted phenyl such as 3-chlorophenyl or methoxyphenyl. In addition, in each of these separate embodiments, one of X₂₅, and X₂₆ is optionally alkyl (such as methyl), nitro, or halo (such as chloro or fluoro) while the remainder of X₁₁, X₂₂, X₃₃, X₄₄ are hydrogen. In addition, in each of these separate embodiments, M₃₄ and M₃₅ are independently optionally hydroxy, alkoxy, thioalkyl, hydrocarbyl or substituted hydrocarbyl.

In an alternative embodiment in which the LXR modulator corresponds to any of Formulae I, IIA, IIB, IIIA, IIIB, or IV, one of M₁₇ and M₁₈ contain a benzene ring or a heteroaromatic moiety. In this embodiment, for example, the LXR modulator may correspond to Formula V:

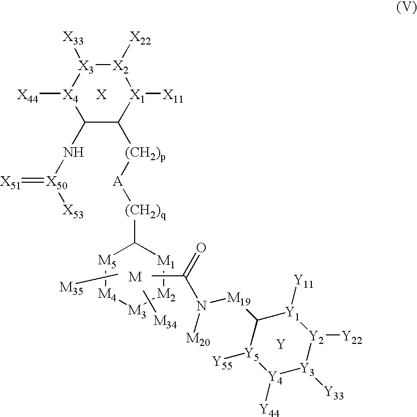

wherein:

the X ring, the M ring and the Y ring are aromatic;

Y₁, Y₂, Y₃, Y₄, and Y₅ are independently a bond, carbon, nitrogen, oxygen or sulfur, provided, however, no more than one of Y₁, Y₂, Y₃, Y₄ and Y₅ is a bond;

Y₁₁, Y₂₂, Y₃₃, Y₄₄, and Y₅₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl; provided, however, Y₁₁, Y₂₂, Y₃₃, Y₄₄ or Y₅₅ is not present when Y₁, Y₂, Y₃ Y₄, or Y₅, respectively, is a bond;

A, M₁, M₂, M₃, M₄, M₅, M₂₀, M₃₄, M₃₅, p, q, X₁, X₂, X₃, X₄, X₁₁, X₂₂, X₃₃, X₄₄, X₅₀, X₅₁, X₅₂, and X₅₃ are as defined in connection with Formula I,

M₁₉ is a bond, hydrocarbyl or substituted hydrocarbyl, and

M₂₀ is hydrogen, hydrocarbyl or substituted hydrocarbyl.

In yet another embodiment, the present invention is directed to compounds corresponding to Formula VI:

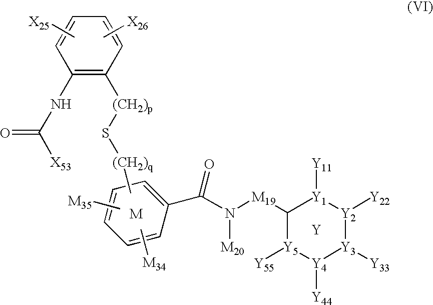

wherein:

the sum of p and q is 1,

M₃₄, M₃₅, and X₅₃ are as defined in connection with Formula I;

M₁₉, M₂₀, Y₁, Y₂, Y₃, Y₄, Y₅, Y₁₁, Y₂₂, Y₃₃, Y₄₄, and Y₅₅ are as defined in connection with Formula V; and

X₂₅ and X₂₆ are independently hydrogen, optionally substituted alkyl, nitro or halo.

In one embodiment in which the LXR modulators correspond to Formula VI, Y₁₁, Y₂₂, Y₃₃, Y₄₄, and Y55 are independently an electron pair, hydrogen, hydrocarbyl, heterosubstituted hydrocarbyl, hydroxy, hydrocarbyloxy, substituted hydrocarbyloxy, mercapto, halo, heterocyclo, cyano, nitro, amino, aminoacyl, thioacyl, acyloxy, or acyl or any adjacent two of Y₁₁, Y₂₂, Y₃₃, Y₄₄, and Y₅₅ together with the atoms to which they are bonded may comprise a fused ring system.

In an alternative embodiment in which the LXR modulator corresponds to any of Formulae I, IIA, IIB, IIIA, IIIB, or IV, M₁₇ and M₁₈ together with the nitrogen atom to which they are attached form a heterocylo. In this embodiment, for example, the LXR modulator corresponds to Formula VII:

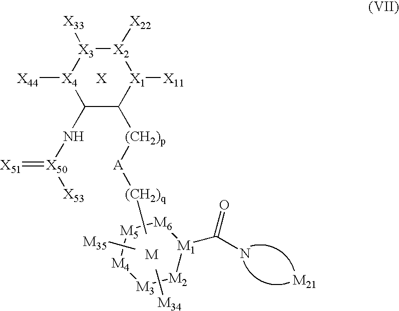

wherein:

the X ring and the M ring are independently aromatic rings;

A, M₁, M₂, M₃, M₄, M₅, M₆, M₃₄, M₃₅, p, q, X₁, X₂, X₃, X₄, X₁₁, X₂₂, X₃₃, X₄₄, X₅₀, X₅₁, X₅₂, and X₅₃ are as defined in connection with Formula I; and

M₂₁ in combination with the nitrogen atom to which it is bonded is heterocyclo.

In a further embodiment the LXR modulators correspond to Formula VIII:

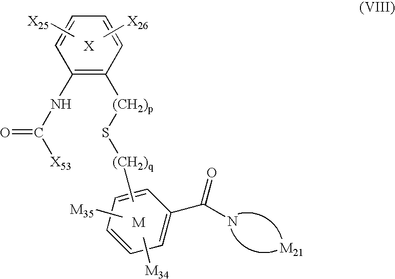

wherein

X₂₅ and X₂₆ are independently hydrogen, optionally substituted alkyl, nitro or halo;

p, q, M₃₄, M₃₅, X₅₃ are as defined in connection with Formula I; and

M₂₁ is as defined in connection with Formula VII.

In one embodiment in which the LXR modulators correspond to Formula VIII, the sum of p and q is 1. For example, p may be 0 and q may be 1. Alternatively, p may be 1 and q may be 0. In addition, in each of these embodiments, the heterocycle comprising M₂₀ may be further substituted by

M₁₉-Y wherein M₁₉ and Y are as defined in connection with Formula VI.

In a further embodiment the LXR modulators correspond to Formula IX:

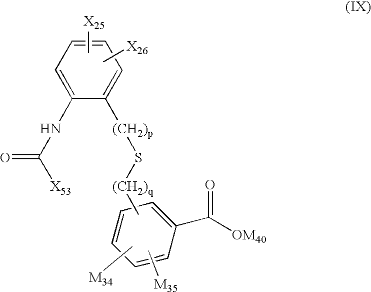

Wherein:

X₂₅ and X₂₆ are independently hydrogen, optionally substituted alkyl, nitro or halo;

p, q, M₃₄, M₃₅, X₅₃ are as defined in connection with Formula l; and

M₄₀ is hydrocarbyl or substituted hydrocarbyl.

In one embodiment in which the LXR modulators correspond to Formula IX, the sum of p and q is 1. For example, p may be 0 and q may be 1. Alternatively, p may be 1 and q may be 0. In addition, in each of these embodiments, M₄₀ may be alkyl or aryl.

Another aspect of the present invention are the prodrugs of the compounds corresponding to the formulae disclosed herein, which are converted under physiological conditions to the biologically active drug by any of a number of chemical and biological mechanisms. In general terms, these prodrug conversion mechanisms are hydrolysis, reduction, oxidation, and elimination.

A further aspect of the invention encompasses conversion of the prodrug to the biologically active drug by elimination of the prodrug moiety. Generally speaking, in this embodiment the prodrug moiety is removed under physiological conditions with a chemical or biological reaction. The elimination results in removal of the prodrug moiety and liberation of the biologically active drug. Any compound of the present invention corresponding to any of the formulas disclosed herein may undergo any combination of the above detailed mechanisms to convert the prodrug to the biologically active compound. For example, a particular compound may undergo hydrolysis, oxidation, elimination, and reduction to convert the prodrug to the biologically active compound. Equally, a particular compound may undergo only one of these mechanisms to convert the prodrug to the biologically active compound.

The compounds of the present invention can exist in tautomeric, geometric or stereoisomeric forms. The present invention contemplates all such compounds, including cis- and trans-geometric isomers, E- and Z-geometric isomers, R- and S-enantiomers, diastereomers, d-isomers, I-isomers, the racemic mixtures thereof and other mixtures thereof, as falling within the scope of any of the formulae disclosed herein. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

Also included in the present invention are the pharmaceutically acceptable salts of any compound having corresponding to any of the formulas disclosed herein and the isomers, tautomers, and prodrugs thereof. The term “pharmaceutically-acceptable salt” includes commonly used to form alkali metal salts and to form addition salts of free acids or free bases. The nature of the salt is not critical, provided that it is pharmaceutically acceptable. Suitable pharmaceutically acceptable acid addition salts of the compounds may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, and galacturonic acid. Suitable pharmaceutically-acceptable base addition salts of the compounds include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procain. All of these salts may be prepared by conventional means from the corresponding compound by reacting, for example, the appropriate acid or base with the selected compound of any of the formulae disclosed herein or the prodrug, isomer, or tautomer thereof.

The present invention also comprises a pharmaceutical composition comprising a therapeutically effective amount of the compound of the invention in association with at least one pharmaceutically acceptable carrier, adjuvant or diluent. Pharmaceutical compositions of the present invention can comprise the active compounds of any of the formulae disclosed herein or the prodrug, isomer, tautomer or prodrug thereof in association with one or more non-toxic, pharmaceutically-acceptable carriers and/or diluents and/or adjuvants (collectively referred to herein as “carrier” materials) and, if desired, other active ingredients. The compostions of the present invention may be administered by any suitable route, preferably in the form of a pharmaceutical composition adapted to such a route, and in a dose effective for the treatment intended.

Synthesis

As depicted in Scheme 1, compounds of the present invention can be prepared by alkylation of (i) to give amine (iii) which can undergo acylation with an acid chloride or anhydride to give the target compounds (iv). Hydrolysis followed by amine coupling give the desired product (v). Additional compounds of the present invention can be prepared as in Scheme 2. Hydrolysis of the compound from Scheme 1 (iii) followed by amine coupling affords the amide (vi). Acylation of this compound gives the desired product (vii). Other compounds of the present invention can be prepared as in Scheme 3. Coupling of the amine with acid (ix) gives amide (x). Alkylation affords the nitro compound (xi). Reduction followed by acylation gives the target compounds (xiii). Additionally sulfides (A₁═S) can be oxidized to the corresponding sulfoxides (A₁═SO) and sulfones (A₁═SO₂).

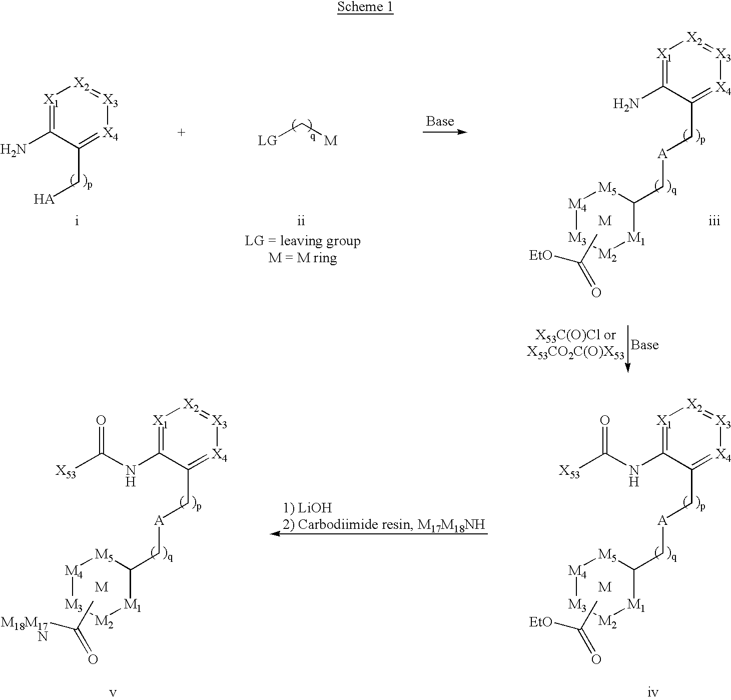

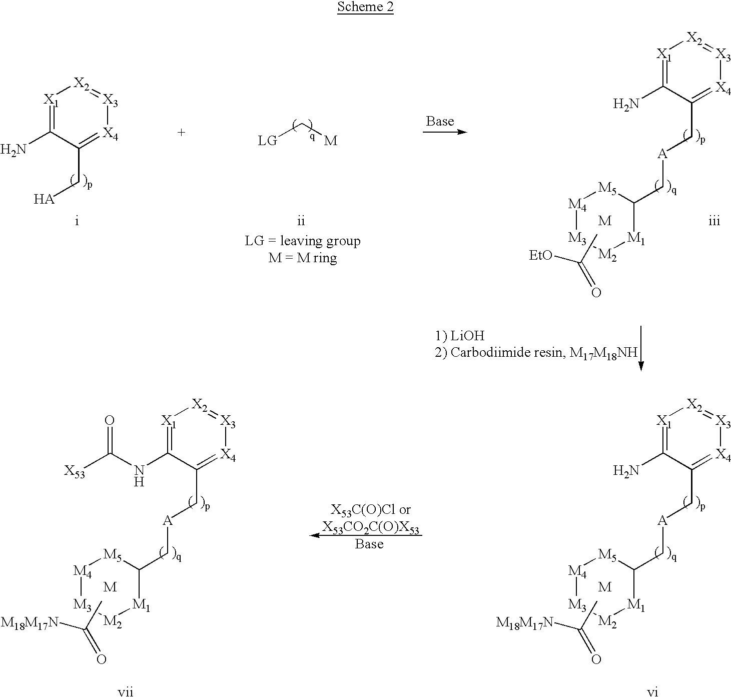

Other compounds of the present invention can be prepared by amine coupling to acid (ix) to form (x) followed by alkylation to yield (xi) (Scheme 3). The nitro group can be reduced to give amine (xii) and subsequent acylation to afford (xiii).

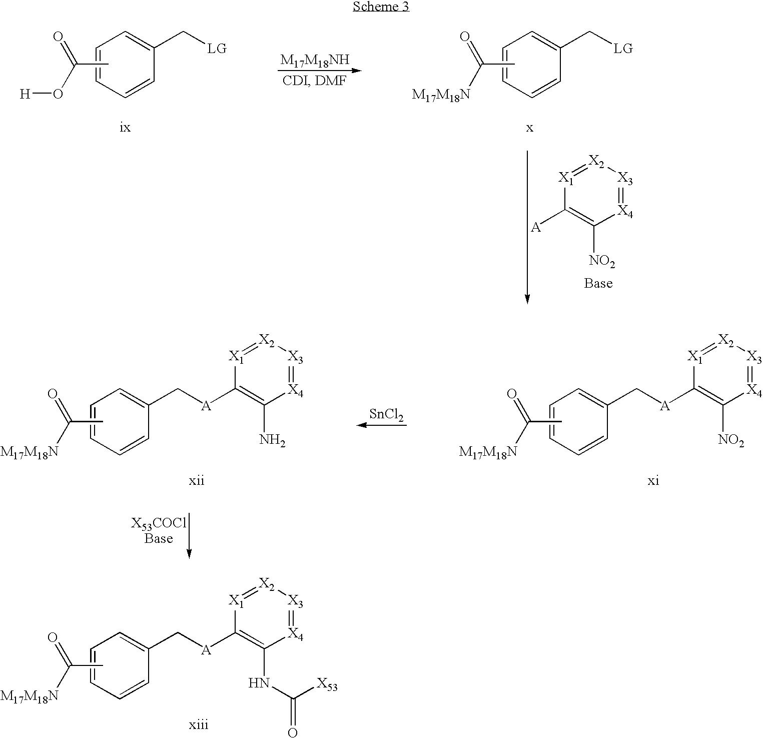

Administration

The LXR modulators useful in the practice of the present invention can be formulated into pharmaceutical compositions and administered by any means that will deliver a therapeutically effective dose. Such compositions can be administered orally, parenterally, intranasally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the LXR modulator will vary depending upon the patient and the particular mode of administration. In general, the pharmaceutical compositions may contain a LXR modulator in the range of about 1 and 2500 mg, more typically, in the range of about 5 and 1000 mg and still more typically, between about 10 and 500 mg. A daily dose of about 0.1 to 50 mg/kg body weight, or more typically, between about 0.1 and about 25 mg/kg body weight and even more typically, from about 0.5 to 10 mg/kg body weight, may be appropriate. The daily dose can be administered in one to about four doses per day. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Definitions

The term “acyl,” as used herein alone or as part of another group, denotes the moiety formed by removal of the hydroxyl group from the —COOH group of an organic carboxylic acid, e.g., RC(O)— wherein R is Rₐ, RₐO—, RₐS—, or RₐR_{b}N—, Rₐ and R_{b} are independently hydrogen, hydrocarbyl, substituted hydrocarbyl, or heterocyclo and “—” is the point of attachment.

The term “acylamino,” as used herein alone or as part of another group, denotes an acyl group as defined above, bonded through a nitrogen atom, e.g., RC(O)N(R_{c})— wherein R is as defined in connection with the term “acyl”, R_{c} is hydrogen, hyrocarbyl, or substituted hydrocarbyl, and “—” denotes the point of attachment.

The term “acyloxy” as used herein alone or as part of another group, denotes an acyl group as defined above, bonded through an oxygen atom

(—O—), e.g., RC(O)O— wherein R is as defined in connection with the term “acyl” and “—” denotes the point of attachment.

The term “acylthio” as used herein alone or as part of another group, denotes an acyl group as defined above, bonded through a sulfur atom (—S—), e.g., RC(O)S— wherein R is as defined in connection with the term “acyl” and “—” denotes the point of attachment.

The term “amino” as used herein alone or as part of another group shall denote a primary, secondary or tertiary amine which may optionally be hydrocarbyl, substituted hydrocarbyl or heteroatom substituted. Specifically included are secondary or tertiary amine nitrogens which are members of a heterocyclic ring. Also specifically included, for example, are secondary or tertiary amino groups substituted by an acyl moiety.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic” shall mean aryl or heteroaromatic.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

The term “heteroaromatic” as used herein alone or as part of another group denote optionally substituted aromatic groups having at least one carbon atom and at least heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “heteroatom” shall mean atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrazolyl, pyrrolyl, indolyl, quinolinyl, thiazolyl, isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic, cyclic or aryl hydrocarbon groups, such as alkaryl, alkenaryl and alkynaryl. Unless otherwise indicated, these moieties preferably comprise 1 to 20 carbon atoms.

The “substituted” alkyl, alkenyl, alkynyl, aryl, hydrocarbyl or heterocyclo moieties described herein are moieties which are substituted with a hydrocarbyl moiety, a substituted hydrocarbyl moiety, a heteroatom, or a heterocyclo. For example, substituents include moieties in which a carbon atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The following examples illustrate the invention.

### EXAMPLES 1-4

Step 1

2-amino thiophenol was dissolved in THF. Methyl (3-bromomethyl) benzoate was added along with PS-DIEA resin and the mixture was agitated at room temperature overnight. The reaction was filtered and the filtrate was concentrated under a stream of nitrogen to afford the product.

Step 2

The product from step 1 was acylated by using excess of the appropriate acid chloride, PS-DMAP resin, PS-DIEA resin in dichloromethane and agitating the reaction overnight at room temperature. PS-Trisamine was added in and the reaction agitated a further 18 h. The reaction was filtered and the filtrate was concentrated under a stream of nitrogen to afford the product.

| Example | | | Mass |
|---|---|---|---|
| Number | Structure | Compound Name(s) | Spec |
| 1 | 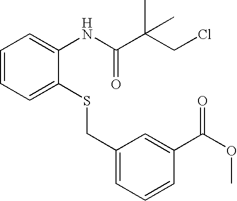 | methyl 3-[({2-[(3-chloro-2,2- dimethylpropanoyl)amino]phenyl}thio)methyl]benzoate | 392.2 |
| 2 | 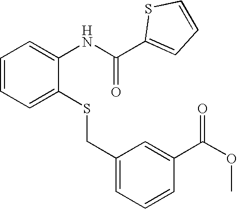 | methyl 3-[({2-[(thien-2-yl carbonyl) amino]phenyl}thio)methyl]benzoate | 384.0 |
| 3 | 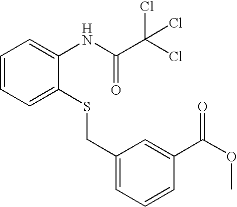 | methyl 3-[({2-[(trichloro acetyl) amino]phenyl}thio)methyl]benzoate | 420.0 |
| 4 | 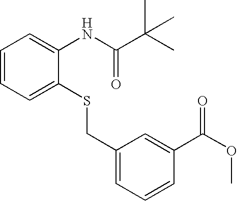 | methyl 3-[({2-[(2,2-dimethyl propanoyl)amino]phenyl}thio)methyl]benzoate | 358.2 |

### EXAMPLES 5-28

Step 1

The product of Example 4 was dissolved in THF and treated with an aqueous solution of lithium hydroxide overnight. The mixture was made acidic with hydrochloric acid and then extracted with ethyl acetate. The organic layer was removed with water then saturated sodium chloride solution. The solvent was removed in vacuo and the residue recrystallized from a mix of ethyl acetate and hexane to afford colorless plates.

Step 2

The product from step 1 was dissolved in dichloromethane and treated with PS-carbodiimide resin and excess of the appropriate amine. The mixture was agitated overnight and then treated with PS-TsOH and MP-carbonate resin, agitated another 24 h filtered and the filtrate concentrated under a stream of nitrogen to afford the products.

| Example | | | Mass |
|---|---|---|---|
| No | Structure | Compound Name(s) | Spec |
| 5 | 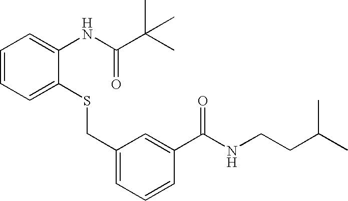 | 3-[({2-[(2,2-dimethylpropanoyl) amino]phenyl}thio) methyl]- N-isopentylbenzamide | 413.2 |
| 6 | 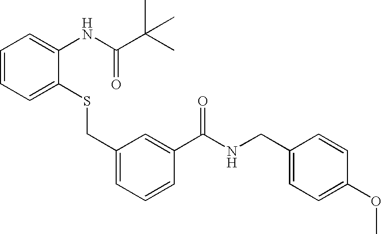 | 3-[8 ({2-[(2,2-dimethylpropanoyl) amino]phenyl}thio)methyl]-N-(4- methoxybenzyl)benzamide | 463.2 |
| 7 | 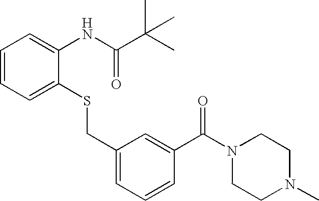 | 2,2-dimethyl-N-[2-({3-[(4-methyl piperazin-1-yl)carbonyl]benzyl}thio) phenyl]propanamide | 426.2 |
| 8 | 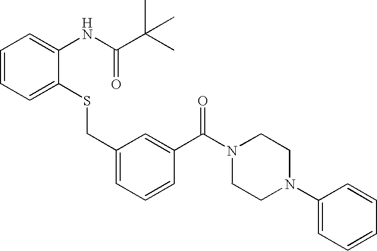 | 2,2-dimethyl-N-[2-({3-[(4-phenyl piperazin-1-yl)carbonyl]benzyl}thio) phenyl]propanamide | |
| 9 | 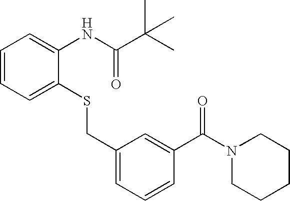 | 2,2-dimethyl-N-(2-{[3-(piperidin- 1-ylcarbonyl) benzyl]thio}phenyl) propanamide | 411.2 |
| 10 | 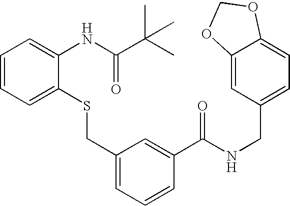 | N-(1,3-benzodioxol-5-yl methyl)- 3-[({2-[(2,2-dimethyl propanoyl)amino]phenyl}thio)methyl]benzamide | 477.2 |
| 11 | 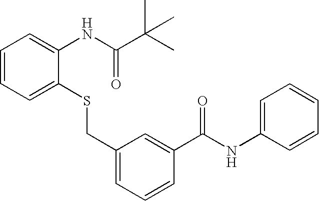 | 3-[({2-[(2,2-dimethyl propanoyl) amino]phenyl}thio)methyl]-N- phenylbenzamide | 419.2 |
| 12 | 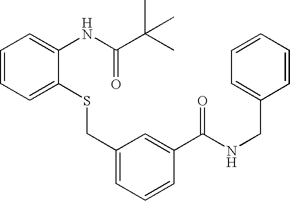 | N-benzyl-3-[({2-[(2,2-dimethyl propanoyl)amino]phenyl}thio) methyl]benazmide | 433.2 |
| 13 | 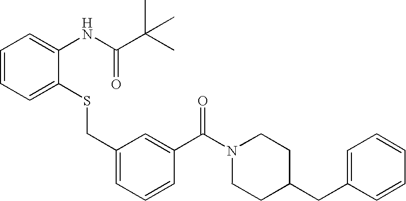 | N-[2-({3-[(4-benzylpiperidin-1- yl)carbonyl]benzyl}thio)phenyl]- 2,2-dimethylpropanamide | 501.2 |
| 14 | 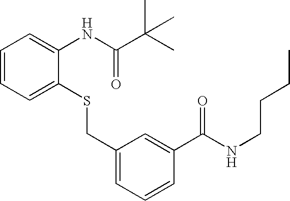 | N-butyl-3-[({2-[(2,2-dimethyl propanoyl)amino]phenyl}thio)me thyl]benzamide | 399.2 |
| 15 | 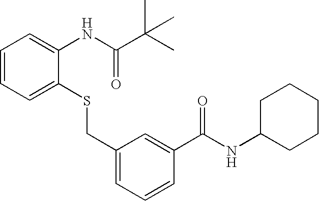 | N-cyclohexyl-3-[({2-[(2,2- dimethylpropanoyl)amino]phenyl}thio)methyl]benzamide | 425.2 |
| 16 | 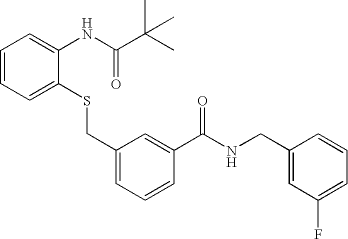 | 3-[({2-[(2,2-dimethyl propa noyl)amino]phenyl}thio)methyl]- N-(3-fluorobenzyl)benzamide | 451.2 |
| 17 | 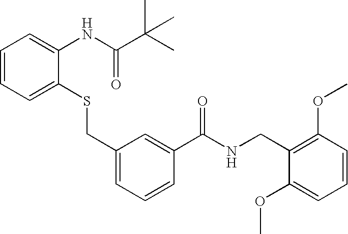 | N-(2,6-dimethoxybenzyl)-3-[({2- [(2,2-dimethylpropanoyl) amino]thio)methyl]benzamide | 493.2 |
| 18 | 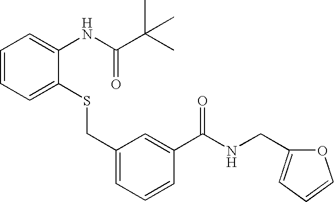 | 3-[({2-[(2,2-dimethylpropanoyl) amino]phenyl}thio)methyl]-N-(2- furylmethyl)benzamide | 423.2 |
| 19 | 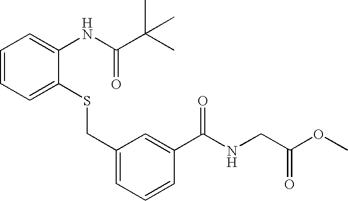 | methyl N-{3-[({2-[(2,2-dimethyl propanoyl)amino]phenyl}thio)me thyl]benzoyl}glycinate | 415.2 |
| 20 | 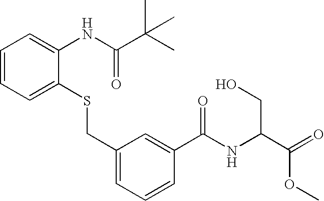 | methyl N-{3-[({2-[(2,2-dimethyl propanoyl)amino]phenyl}thio)me thyl]benzoyl}serinate | 445.2 |
| 21 | 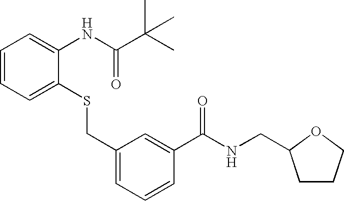 | 3-[({2-[(2,2-dimethylpropanoyl) amino]phenyl}thio)methyl]N-(tetrahydrofuran-2-ylmethyl) benzamide | 427.2 |
| 22 | 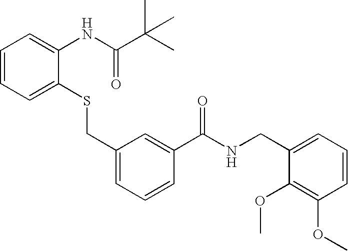 | N-(2,3-dimethoxybenzyl)-3-[({2- [(2,2-dimethylpropanoyl) benzamide | 493.2 |
| 23 | 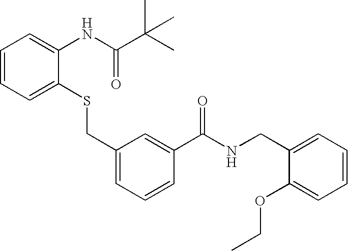 | 3-[({2-[(2,2-dimethylpropanoyl) amino]phenyl}thio)methyl]-N-(2- ethoxybenzyl)benzamide | 477.2 |
| 24 | 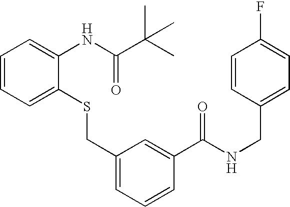 | 3-[({2-[(2,2-dimethylpropanoyl) amino]phenyl}thio)methyl]-N-(4- fluorobenzyl)benzamide | 451.2 |
| 25 | 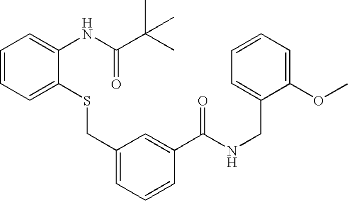 | 3-[({2-[(2,2-dimethylpropanoyl) amino]phenyl}thio)methyl]-N-(2- methoxybenzyl)benzamide | 463.2 |
| 26 | 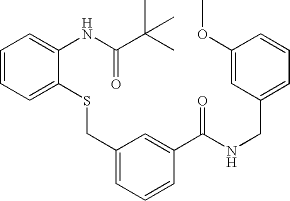 | 3-[({2-[(2,2-dimethylpropanoyl) amino]phenyl}thio)methyl]-N-(3- methoxybenzyl)benzamide | 463.2 |
| 27 | 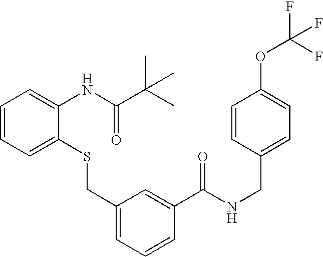 | 3-[({2-[(2,2-dimethylpropanoyl) amino]phenyl}thio)methyl]-N-[4- (trifluoromethoxy)benzyl]benzamide | 517.2 |
| 28 | 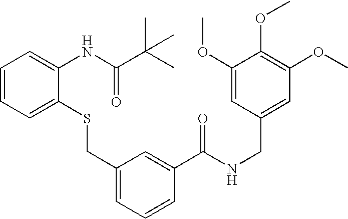 | 3-[({2-[(2,2-dimethylpropanoyl) amino]phenyl}thio)methyl]-N- (3,4,5-trimethoxybenzyl) benzamide | 523.2 |
| 29 | 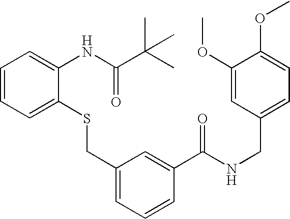 | N-(3,4-dimethoxybenzyl)-3-[({2- [(2,2-dimethylpropanoyl)amino]phenyl}thio)methyl]benzamide | 493.2 |
| 30 | 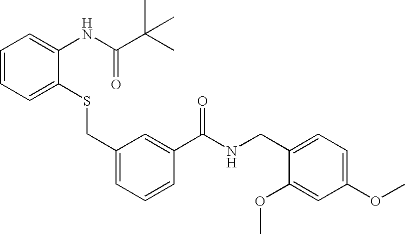 | N-(2,4-dimethoxybenzyl)-3-[({2- [(2,2-dimethylpropanoyl)amino]phenyl}thio)methyl]benzamide | 493.2 |

### EXAMPLES 31-38

Step 1

2-amino thiophenol was dissolved in THF. Methyl (3-bromomethyl) benzoate was added along with PS-DIEA resin and the mixture was agitated at room temperature overnight. The reaction was filtered and the filtrate was concentrated under a stream of nitrogen to afford the product.

Step 2

The product of step 1 was dissolved in THF and treated with an aqueous solution of lithium hydroxide overnight. The mixture was made neutral with hydrochloric acid and then extracted with ethyl acetate. The organic layer was washed with water and then with saturated sodium chloride solution. The solvent was removed in vacuo to afford the product.

Step 3

The product from step 2 was dissolved in dichloromethane and treated with PS-carbodiimide resin and the appropriate amine. The mixture was agitated overnight and then treated with MP-carbonate resin, agitated another 24 h filtered and the filtrate concentrated under a stream of nitrogen to afford the products.

Step 4

The product from step 3 was acylated by using excess of the appropriate acid chloride, PS-DMAP resin, PS-DIEA resin in dichloromethane and agitating the reaction overnight at room temperature. PS-Trisamine was added in and the reaction agitated a further 18 h. The reaction was filtered and the filtrate was concentrated under a stream of nitrogen to afford the product.

| Example | | | Mass |
|---|---|---|---|
| No | Structure | Compound Name(s) | Spec |
| 31 | 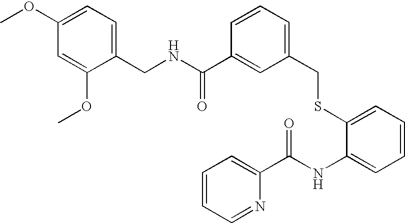 | N-{2-[(3-{[(2,4-dimethoxybenzyl) amino]carbonyl}benzyl)thio]phenyl}pyridine-2-carboxamide | 514.2 |
| 32 | 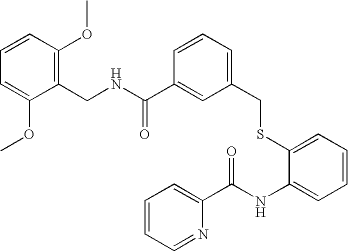 | N-{2-[(3-{[(2,6-dimethoxybenzyl) amino]carbonyl}benzyl)thio]phen yl}pyridine-2-carboxamide | 514.2 |
| 33 | 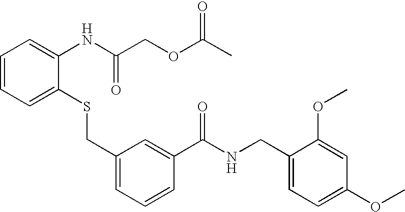 | 2-({2-[(3-{[(2,4-dimethoxybenzyl) amino]carbonyl}benzyl)thio]phen yl}amino)-2-oxoethyl acetate | 509.2 |
| 34 | 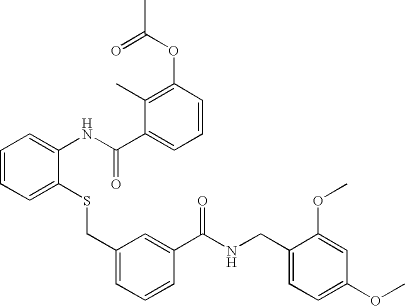 | 3-[({2-[(3-{[(2,4- dimethoxybenzyl) amino]carbonyl}benzyl)thio]phen yl}amino)carbonyl]-2- methylphenyl acetate | 585.2 |
| 35 | 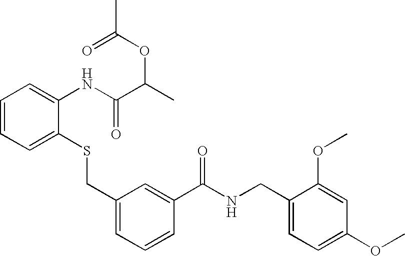 | 2-({2-[(3-{[(2,4-dimethoxybenzyl) amino]carbonyl}benzyl)thio]phen yl}amino)-1-methyl-2-oxoethyl acetate | 523.2 |
| 36 | 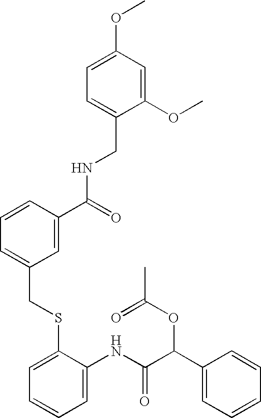 | 2-({2-[(3-{[(2,4-dimethoxybenzyl) amino]carbonyl}benzyl)thio]phen yl}amino)-2-oxo-1-phenylethyl acetate | 585.2 |
| 37 | 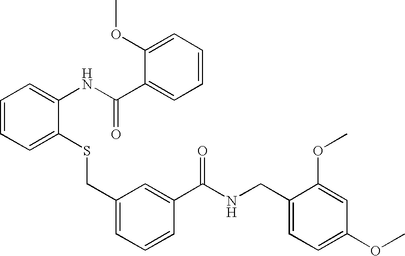 | N-{2-[(3-{[(2,4-dimethoxybenzyl) amino]carbonyl}benzyl)thio]phen yl}-2-methoxybenzamide | 543.2 |
| 38 | 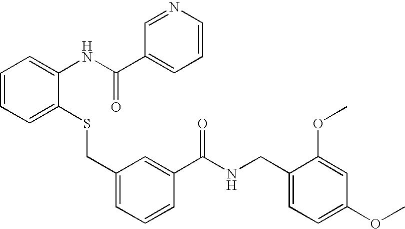 | N-{2-[(3-{[(2,4-dimethoxybenzyl) amino]carbonyl}benzyl)thio]phen yl}nicotinamide | 514.2 |

### EXAMPLES 39-41

Step 1

2-amino thiophenol was dissolved in THF. Methyl (3-bromomethyl) benzoate was added along with PS-DIEA resin and the mixture was agitated at room temperature overnight. The reaction was filtered and the filtrate was removed under a stream of Nitrogen to afford the product.

Step 2

The product of step 1 was dissolved in THF and treated with an aqueous solution of lithium hydroxide overnight. The mixture was made neutral with hydrochloric acid and then extracted with ethyl acetate. The organic layer was washed with water then saturated sodium chloride solution. The solvent was removed in vacuo to afford the product.

Step 3

The product from step 2 was dissolved in dichloromethane and treated with PS-carbodiimide resin and the appropriate amine. The mixture was agitated overnight and then treated with MP-carbonate resin, agitated another 24 h filtered and the filtrate concentrated under a stream of nitrogen to afford the products.

Step 4

The product from step 3 was then acylated by using excess of chloromethyl acetyl chloride, PS-DMAP resin, PS-DIEA in dichloromethane and agitating the reaction overnight at room temperature. The reaction was filtered and the filtrate was reduced under a stream of nitrogen to afford the product.

Step 5

The product of step 4 was combined in dichloromethane (2.5 mL) with an excess of the appropriate amine and DIEA resin (4 equivalents). The reaction was subjected to microwave heating for 25 minutes at 100 degrees filtered and the residue chromatographed on silica to give the title product.

### EXAMPLES 39-41

| Example | | | Mass |
|---|---|---|---|
| No | Structure | Compound Name(s) | Spec |
| 39 | 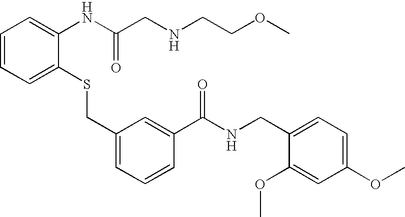 | N-(2,4-dimethoxybenzyl)-3-{[(2- {[N-(2-methoxyethyl)glycyl]amino}phenyl)thio]methyl}benzamide | 524.2 |
| 40 | 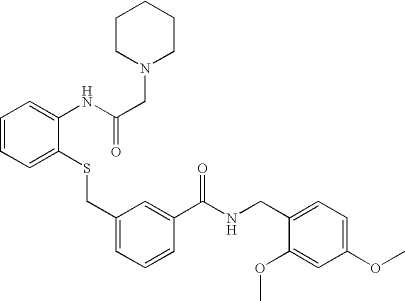 | N-(2,4-dimethoxybenzyl)-3-[({2- [(piperidin-1-ylacetyl)amino]phenyl}thio)methyl]benzamide | 534.2 |
| 41 | 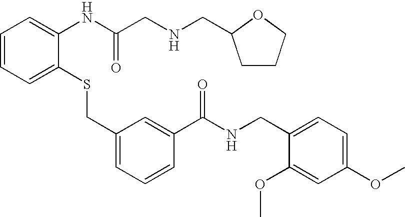 | N-(2,4-dimethoxybenzyl)-3-{[(2- {[N-(tetrahydrofuran-2-ylmethyl) glycyl]amino}phenyl)thio]methyl}benzamide | 550.2 |

### EXAMPLE 42

N-(2,4-dimethoxybenzyl)-3-[({3-[(2,2-dimethylpropanoyl)amino]pyridin-2-yl}thio)methyl] benzamide

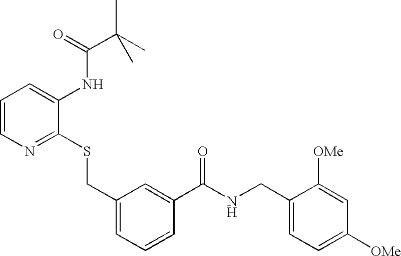

Step 1

3-chloromethylbenzoic acid (5.9 mmol) was dissolved in DMF and carbonyldiimidazole (6.2 mmol) added. After 5 minutes the amine (5.8 mmol) was added and the reaction stirred at room temperature for 4 h. The reaction mixture was diluted with water and extracted with dichloromethane. The combined organics were dried over MgSO₄, filtered, and the solvent removed. The crude residue was purified by flash chromatography.

Step 2

The product from step 1 (1.6 mmol) was dissolved in DMF along with the nitrothiopyridine (1.6 mmol). To this solution was added Hunig's base (3.1 mmol) and the reaction stirred at room temperature overnight. The reaction mixture was treated with acetic acid solution and the product extracted with dichloromethane. The combined organics were dried over MgSO₄, filtered, and the solvent removed. The crude residue was purified by flash chromatography.

Step 3

The product from step 2 (0.36 mmol) was dissolved in ethanol and tin chloride dihydrate added. The reaction mixture was heated at 70° C for 2 h. The residue was diluted with water and the product extracted with DCM. The combined organics were dried over MgSO₄, filtered, and the solvent removed. The crude product was used in the next reaction.

Step 4

The product from step 3 (0.07 mmol) was dissolved in DCM and the acid chloride (0.08 mmol) added. To this was added PS-DIEA and PS-DMAP and the reaction stirred at RT overnight. The crude product was filtered and the solvent removed. The product was purified by flash chromatography.

### EXAMPLES 43-54

| Example | | | Formula |
|---|---|---|---|
| No | Structure | Compound Name(s) | weight |
| 43 | 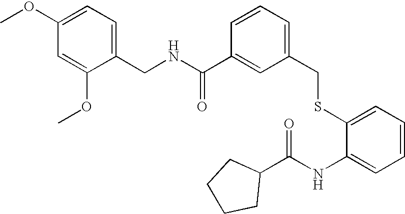 | 3-[({2-[(cyclopentylcarbonyl) amino]phenyl}thio)methyl]-N-(2,4- dimethoxybenzyl)benzamide | 504.65 |
| 44 | 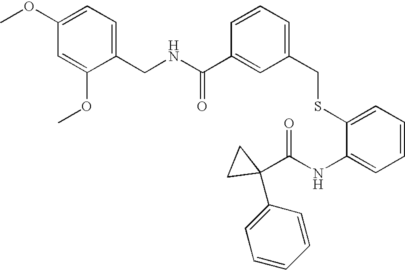 | N-(2,4-dimethoxybenzyl)-3-{[(2-{[(1- phenylcyclopropyl)carbonyl]amino}phenyl)thio]methyl}benzamide | 552.7 |
| 45 | 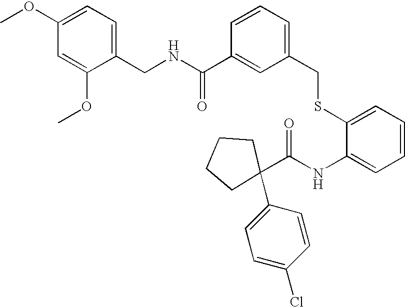 | 3-({[2-({[1-(4- chlorophenyl)cyclopentyl]carbonyl}amino)phenyl]thio}methyl)-N-(2,4- dimethoxybenzyl)benzamide | 615.2 |
| 46 | 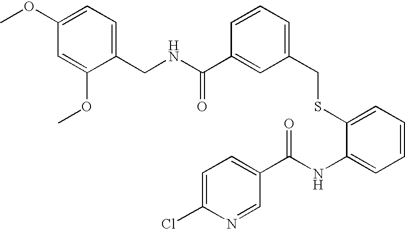 | 6-chloro-N-{2-[(3-{[(2,4-dimethoxy benzyl)amino]carbonyl}benzylthio]phenyl}nicotinamide | 548.07 |
| 47 | 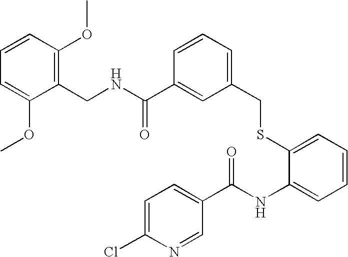 | 6-chloro-N-{2-[(3-{[(2,6-dimethoxy benzyl)amino]carbonyl}benzyl)thio]phenyl}nicotinamide | 548.07 |
| 48 | 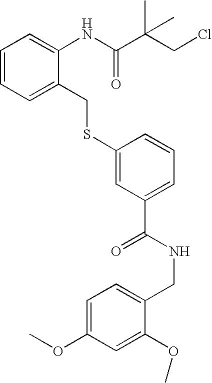 | 3-({2-[(3-chloro-2,2-dimethyl propanoyl)amino]benzyl}thio)-N- (2,4-dimethoxybenzyl)benzamide | 527.09 |
| 49 | 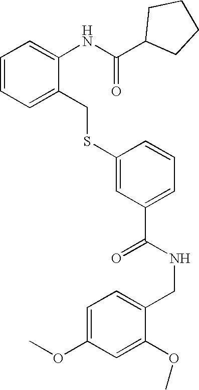 | 3-({2-[(cyclopentylcarbonyl)amino]benzyl}thio)-N-(2,4-dimethoxy benzyl)benzamide | 504.65 |
| 50 | 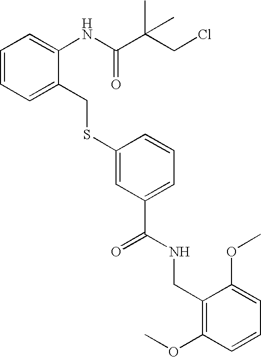 | 3-({2-[(3-chloro-2,2-dimethyl propanoyl)amino]benzyl}thio)-N- (2,6-dimethoxybenzyl)benzamide | 527.09 |
| 51 | 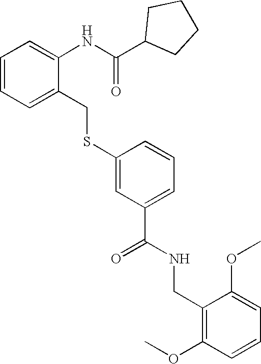 | 3-({2-[(cyclopentylcarbonyl) amino]benzyl}thio)-N-(2,6- dimethoxybenzyl)benzamide | 504.65 |
| 52 | 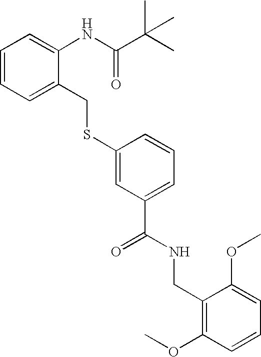 | N-(2,6-dimethoxybenzyl)-3-({2-[(2,2- dimethylpropanoyl)amino]benzyl}thio)benzamide | 492.64 |
| 53 | 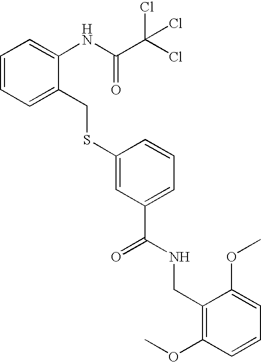 | N-(2,6-dimethoxybenzyl)-3-({2- [(trichloroacetyl)amino]benzyl}thio)b enzamide | 553.9 |
| 54 | 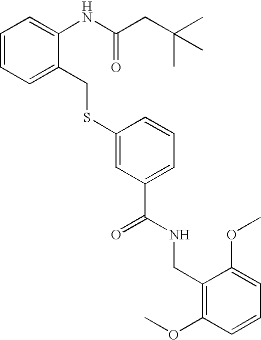 | N-(2,6-dimethoxybenzyl)-3-({2-[(3,3- dimethylbutanoyl)amino]benzyl}thio) benzamide | 506.67 |

### EXAMPLE 55

LXR Reporter Gene Transactivation Assay for High-Throughput Screen

Human hepatic cells (Huh-7) were cotransfected with a luciferase reporter gene (pGal4-RE), where transcription of luciferase gene is driven by the Gal4 response element, and a chimeric gene construct of liver X receptor (Gal4_{DBD}-LXRα_{LBD}), which comprises a DNA sequence that encodes a hybrid protein of LXR ligand binding domain (LXR_{LBD}) and Gal4 DNA-binding domain (Gal4_{DBD}). The transfection was performed in culture dishes using LipofectAMINE2000 reagent. The transfected cells were harvested 20 hr later and resuspended in assay medium containing RPMI 1640 medium, 2% fetal bovine lipoprotein deficient serum, 100 units/ml pencillin and 100 μg/ml streptomycin.

In screening for LXR modulators, the transfected cells were dispensed in an assay plate (384-well white tissue culture plate) containing the test compounds at 10 μM final concentration and incubated for 24 hr. The effects of test compounds on the activation of LXR_{LBD} and hence luciferase transcription was determined by measuring the luciferase activity using Steady-Glo luciferase assay substrate. Luciferase activity results are expressed as the fold-induction relative to DMSO controls. Compounds that exhibited >10 fold induction were then retested and the EC₅₀ was determined as the concentration necessary to produce 50% of the maximal luciferase activity. Each of the compounds of Examples 1-54 was found to have an EC₅₀ of less than 50 μM.

What is claimed is:

1. A compound corresponding to Formula I and the isomers, tautomers, salts and prodrugs thereof: 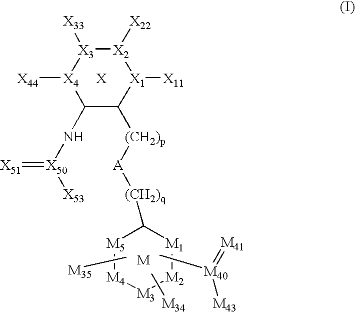
wherein:
the X ring and the M ring are independently aromatic rings;
A is oxygen, sulfur, sulfoxide, sulfone, —NHC(═A₂)— or —C(═A₂)NH—;
A₂ is oxygen or sulfur;
M₁, M₂, M₃, M₄, and M₅ are independently a bond, carbon, nitrogen, oxygen or sulfur, provided, however, no more than one of M₁, M₂, M₃, M₄, and M₅ is a bond;
M₃₄ and M₃₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl, or M₃₄ and M₃₅ are bonded to adjacent carbon atoms and together with the atoms to which they are bonded form a fused ring system;
M₄₀ is carbon, sulfur or sulfoxide;
M₄₁ is oxygen, sulfur, or NM₄₂;
M₄₂ is hydrogen, hydrocarbyl, or substituted hydrocarbyl; and
M₄₃ is hydrogen, hydrocarbyl, substituted hydrocarbyl, hydrocarbyloxy, substituted hydrocarbyloxy, amino, hydrocarbylthio, or substituted hydrocarbylthio;
p and q are independently 0, 1,or 2;
X₁, X₂, X₃, and X₄ are independently a bond, carbon, nitrogen, oxygen or sulfur, provided, however, no more than one of X₁, X₂, X₃, and X₄ is a bond;
X₁₁, X₂₂, X₃₃, and X₄₄, are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl; provided, however, X₁₁, X₂₂, X₃₃, or X₄₄ is not present when X₁, X₂, X₃ or X₄, respectively, is a bond;
X₅₀ is carbon, sulfur or sulfoxide,
X₅₁ is oxygen, sulfur, or NX₅₂,
X₅₂ is hydrogen, hydrocarbyl, or substituted hydrocarbyl; and
X₅₃ is hydrogen, hydrocarbyl, substituted hydrocarbyl, heterocyclo, or amino.

2. The compound of claim 1 wherein the sum of p and q is 1.

3. The compound of claim 1 wherein X₅₀ is carbon and X₅₁ is oxygen.

4. The compound of claim 1 wherein X₅₃ is heterocyclo, optionally substituted alkyl, or optionally substituted phenyl.

5. The compound of claim 1 wherein X₁₁, X₂₂, X₃₃, and X₄₄ are hydrogen.

6. The compound of claim 2 wherein each of X₁-X₄ and M₁-M₅ is carbon.

7. A compound corresponding to Formula IV and the isomers, tautomers, salts and prodrugs thereof: 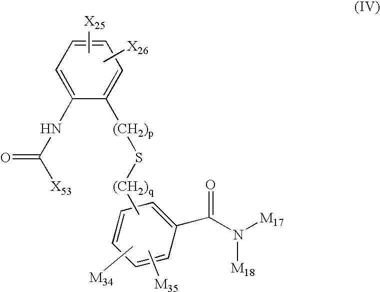
wherein:
M₁₇ is hydrogen, hydrocarbyl, substituted hydrocarbyl, hydrocarbyloxy, heterocyclo, amino, or acyl;
M₁₈ is hydrocarbyl, substituted hydrocarbyl, or heterocyclo;
M₃₄ and M₃₅ are independently hydrogen, hydrocarbyl, substituted hydrocarbyl, amino, alkoxy, halogen, or nitro;
p and q are independently 0, 1,or 2;
X₂₅ and X₂₆ are independently hydrogen, optionally substituted alkyl, nitro or halo, and
X₅₃ is hydrocarbyl, substituted hydrocarbyl or heterocyclo.

8. A compound corresponding to Formula V and the isomers, tautomers, salts and prodrugs thereof: 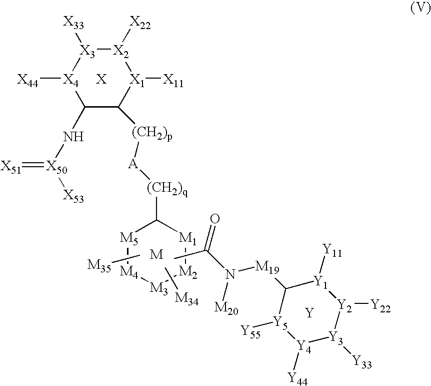
wherein:
the X ring, the M ring and the Y ring are independently aromatic;
A is oxygen, sulfur, sulfoxide, sulfone, —NHC(═A₂)— or —C(═A₂)NH—;
A₂ is oxygen or sulfur;
M₁, M₂, M₃, M₄, and M₅ are independently a bond, carbon, nitrogen, oxygen or sulfur, provided, however, no more than one of M₁, M₂, M₃, M₄, and M₅ is a bond;
M₁₉ is a bond, hydrocarbyl or substituted hydrocarbyl;
M₂₀ is hydrogen, hydrocarbyl, substituted hydrocarbyl, or heterocyclo;
M₃₄ and M₃₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl, or M₃₄ and M₃₅ are bonded to adjacent carbon atoms and together with the atoms to which they are bonded form a fused ring system;
p and q are independently 0, 1,or 2;
X₁, X₂, X₃, and X₄ are independently a bond, carbon, nitrogen, oxygen or sulfur, provided, however, no more than one of X₁, X₂, X₃, and X₄ is a bond;
X₁₁, X₂₂, X₃₃, and X₄₄, are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl; provided, however, X₁₁, X₂₂, X₃₃, or X₄₄ is not present when X₁, X₂, X₃ or X₄, respectively, is a bond;
X₅₀ is carbon, sulfur or sulfoxide;
X₅₁ is oxygen, sulfur, or NX₅₂;
X₅₂ is hydrogen, hydrocarbyl, or substituted hydrocarbyl;
X₅₃ is hydrogen, hydrocarbyl, substituted hydrocarbyl, heterocyclo, or amino;
Y₁, Y₂, Y₃, Y₄, and Y₅ are independently a bond, carbon, nitrogen, oxygen or sulfur, provided, however, no more than one of Y₁, Y₂, Y₃, Y₄ and Y₅ is a bond; and
Y₁₁, Y₂₂, Y₃₃, Y₄₄, and Y₅₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl, or one of Y11 and Y₂₂, Y₂₂ and Y₃₃ or Y₃₃ and Y₄₄ and Y₄₄ and Y₅₅ and the atoms to which they are attached form a fused ring; provided, however, Y₁₁, Y₂₂, Y₃₃, Y₄₄ or Y₅₅ is not present when Y₁, Y₂, Y₃ Y₄, or Y₅, respectively, is a bond.

9. A compound corresponding to Formula VI and the isomers, tautomers, salts and prodrugs thereof: 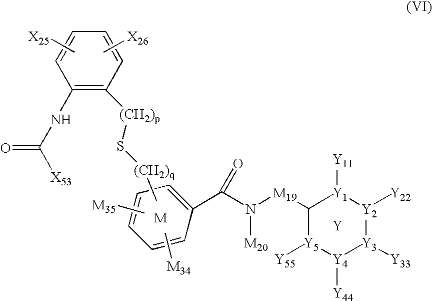
wherein:
M₁₉ is a bond, hydrocarbyl or substituted hydrocarbyl;
M₂₀ is hydrogen, hydrocarbyl, substituted hydrocarbyl, or heterocyclo;
M₃₄ and M₃₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl, or M₃₄ and M₃₅ are bonded to adjacent carbon atoms and together with the atoms to which they are bonded form a fused ring system;
the sum of p and q is 1;
X₂₅ and X₂₆ are independently hydrogen, optionally substituted alkyl, nitro or halo;
X₅₃ is hydrogen, hydrocarbyl, substituted hydrocarbyl, heterocyclo, or amino;
Y₁, Y₂, Y₃, Y₄, and Y₅ are independently a bond, carbon, nitrogen, oxygen or sulfur, provided, however, no more than one of Y₁, Y₂, Y₃, Y₄ and Y₅ is a bond; and
Y₁₁, Y₂₂, Y₃₃, Y₄₄, and Y₅₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl, or one of Y₁₁, and Y₂₂, Y₂₂ and Y₃₃ or Y₃₃ and Y₄₄ and Y₄₄ and Y₅₅ and the atoms to which they are attached form a fused ring; provided, however, Y₁₁, Y₂₂, Y₃₃, Y₄₄ or Y₅₅ is not present when Y₁, Y₂, Y₃ Y₄, or Y₅, respectively, is a bond.

10. The compound of claim 9 wherein M₁₉ is methylene.

11. The compound of claim 9 wherein M₂₀ is hydrogen.

12. The compound of claim 9 wherein X₅₃ is heterocyclo, optionally substituted alkyl, or optionally substituted phenyl.

13. The compound of claim 9 wherein M₁₉ is methylene; at least one of M₂₀, M₃₄ and M₃₅ is alkoxy, nitro, or halo; one of X₂₅, X₂₆, is hydrogen and the other is an optionally substituted alkyl, nitro, or halo; and Y₁-Y₅ are carbon.

14. The compound of claim 9 wherein M₁₉ is methylene; X₂₅, X₂₆, M₂₀, M₃₄ and M₃₅ are hydrogen; and Y₁-Y₅ are carbon.

15. The compound of claim 14 wherein any two of Y₁₁, Y₃₃, and Y₅₅ are alkoxy.

16. The compound of claim 15 wherein the alkoxy is methoxy.

17. A compound corresponding to Formula VII and the isomers, tautomers, salts and prodrugs thereof: 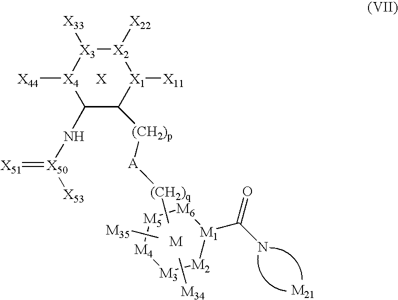
wherein:
the X ring and the M ring are independently aromatic rings;
A is oxygen, sulfur, sulfoxide, sulfone, —NHC(═A₂)— or —C(═A₂)NH—;
A₂ is oxygen or sulfur;
M₁, M₂, M₃, M₄, M₅, and M₆, are independently a bond, carbon, nitrogen, oxygen or sulfur, provided, however, no more than one of M₁, M₂, M₃, M₄, M₅, and M₆, is a bond;
M₂₁ in combination with the nitrogen atom to which it is bonded form a heterocylcic ring;
M₃₄ and M₃₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl, or M₃₄ and M₃₅ are bonded to adjacent carbon atoms and together with the atoms to which they are bonded form a fused ring system;
p and q are independently 0, 1,or 2;
X₁, X₂, X₃, and X₄ are independently a bond, carbon, nitrogen, oxygen or sulfur, provided, however, no more than one of X₁, X₂, X₃, and X₄ is a bond;
X₁₁, X₂₂, X₃₃, and X₄₄, are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl; provided, however, X₁₁, X₂₂, X₃₃, or X₄₄ is not present when X₁, X₂, X₃ or X₄, respectively, is a bond;
X₅₀ is carbon, sulfur or sulfoxide;
X₅₁ is oxygen, sulfur, or NX₅₂;
X₅₂ is hydrogen, hydrocarbyl, or substituted hydrocarbyl; and
X₅₃ is hydrogen, hydrocarbyl, substituted hydrocarbyl, heterocyclo, or amino.

18. A compound corresponding to Formula VIII and the isomers, tautomers, salts and prodrugs thereof: 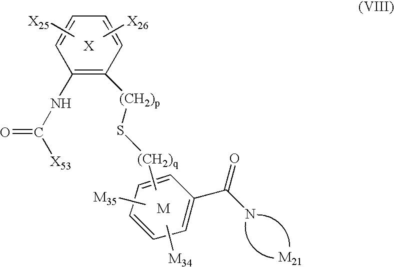
wherein;
M₂₁ in combination with the nitrogen atom to which it is bonded form a heterocylcic ring;
M₃₄ and M₃₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl, or M₃₄ and M₃₅ are bonded to adjacent carbon atoms and together with the atoms to which they are bonded form a fused ring system;
p and q are independently 0, 1,or 2;
X₂₅ and X₂₆ are independently hydrogen, optionally substituted alkyl, nitro or halo; and
X₅₃ is hydrogen, hydrocarbyl, substituted hydrocarbyl, heterocyclo, or amino.

19. The compound of claim 18 wherein the sum of p and q is 1.

20. The compound of claim 18 wherein X₅₃ is heterocyclo, optionally substituted alkyl, or optionally substituted phenyl.

21. The compound of claim 18 wherein one of X₂₅ and X₂₆ is an optionally substituted alkyl, nitro or halo, and the other is hydrogen.

22. The compound of claim 18 wherein X₂₅, X₂₆, M₃₄ and M₃₅ are hydrogen.

23. A compound corresponding to Formula IX and the isomers, tautomers, salts and prodrugs thereof: 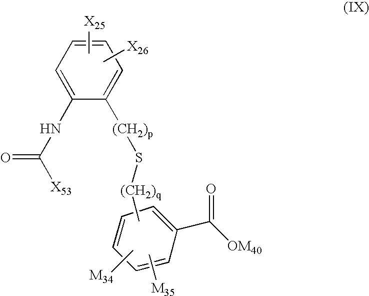
wherein;
M₃₄ and M₃₅ are independently an electron pair, hydrogen, hydrocarbyl, substituted hydrocarbyl, hydroxy, hydrocarbyloxy, substituted hydrocarbyloxy, mercapto, halo, heterocyclo, cyano, nitro, amino, acyloxy, or acyl, or M₃₄ and M₃₅ are bonded to adjacent carbon atoms and together with the atoms to which they are bonded form a fused ring system;
M₄₀ is hydrocarbyl or substituted hydrocarbyl;
p and q are independently 0, 1,or 2;
X₂₅ and X₂₆ are independently hydrogen, optionally substituted alkyl, nitro or halo; and
X₅₃ is hydrogen, hydrocarbyl, substituted hydrocarbyl, heterocyclo, or amino.

24. The compound of claim 23 wherein the sum of p and q is 1.

25. The compound of claim 23 wherein one of X₂₅ and X₂₆ is an optionally substituted alkyl, nitro or halo, and the other is hydrogen.

26. The compound of claim 23 wherein X₂₅, X₂₆, M₃₄ and M₃₅ are hydrogen; and M₄₀ is methyl.

27. A compound selected from the group consisting of
methyl 3-[({2-[(3-chloro-2,2-dimethylpropanoyl)amino]phenyl}thio)methyl]benzoate;
methyl 3-[({2-[(thien-2-ylcarbonyl)amino]phenyl}thio)methyl]benzoate;
methyl 3-[({2-[(trichloroacetyl)amino]phenyl}thio)methyl]benzoate;
methyl 3-[({2-[(2,2-dimethylpropanoyl)amino]phenyl}thio)methyl]benzoate;
3-[({2-[(2,2-dimethylpropanoyl)amino]phenyl}thio)methyl]-N-isopentyl benzamide;
3-[({2-[(2,2-dimethylpropanoyl)amino]phenyl}thio)methyl]-N-(4-methoxy benzyl) benzamide;
2,2-dimethyl-N-[2-({3-[(4-methylpiperazin-1-yl)carbonyl]benzyl}thio)phenyl]propanamide;
2,2-dimethyl-N-[2-({3-[(4-phenylpiperazin-1-yl)carbonyl]benzyl}thio)phenyl]propanamide;
2,2-dimethyl-N-(2-{[3-(piperidin-1-ylcarbonyl)benzyl]thio}phenyl) propanamide;
N-(1,3-benzodioxol-5-ylmethyl)-3-[({2-[(2,2-dimethylpropanoyl)amino]phenyl}thio)methyl]benzamide;
3-[({2-[(2,2-dimethylpropanoyl)amino]phenyl}thio)methyl]-N-phenyl benzamide;
N-benzyl-3-[({2-[(2,2-dimethylpropanoyl)amino]phenyl}thio)methyl]benzamide;
N-[2-({3-[(4-benzylpiperidin-1-yl)carbonyl]benzyl}thio)phenyl]-2,2-dimethylpropanamide;
N-butyl-3-[({2-[(2,2-dimethylpropanoyl)amino]phenyl}thio)methyl]benzamide;
N-cyclohexyl-3-[({2-[(2,2-dimethylpropanoyl)amino]phenyl}thio)methyl]benzamide;
3-[({2-[(2,2-dimethylpropanoyl)amino]phenyl}thio)methyl]-N-(3-fluoro benzyl)benzamide;
N-(2,6-dimethoxybenzyl)-3-[({2-[(2,2-dimethylpropanoyl)amino]phenyl}thio) methyl]benzamide;
3-[({2-[(2,2-dimethylpropanoyl)amino]phenyl}thio)methyl]-N-(2-furylmethyl) benzamide;
methyl N-{3-[({2-[(2,2-dimethylpropanoyl)amino]phenyl}thio)methyl]benzoyl}glycinate;
methyl N-{3-[({2-[(2,2-dimethylpropanoyl)amino]phenyl}thio)methyl]benzoyl}serinate;
3-[({2-[(2,2-dimethylpropanoyl)amino]phenyl}thio)methyl]-N-(tetrahydro furan-2-ylmethyl)benzamide;
N-(2,3-dimethoxybenzyl)-3-[({2-[(2,2-dimethylpropanoyl)amino]phenyl}thio)methyl]benzamide;
3-[({2-[(2,2-dimethylpropanoyl)amino]phenyl}thio)methyl]-N-(2-ethoxy benzyl)benzamide;
3-[({2-[(2,2-dimethylpropanoyl)amino]phenyl}thio)methyl]-N-(4-fluoro benzyl)benzamide;
3-[({2-[(2,2-dimethylpropanoyl)amino]phenyl}thio)methyl]-N-(2-methoxy benzyl)benzamide;
3-[({2-[(2,2-dimethylpropanoyl)amino]phenyl}thio)methyl]-N-(3-methoxy benzyl)benzamide;
3-[({2-[(2,2-dimethylpropanoyl)amino]phenyl}thio)methyl]-N-[4-(trifluoro methoxy)benzyl]benzamide;
3-[({2-[(2,2-dimethylpropanoyl)amino]phenyl}thio)methyl]-N-(3,4,5-trimethoxybenzyl)benzamide;
N-(3,4-dimethoxybenzyl)-3-[({2-[(2,2-dimethylpropanoyl)amino]phenyl}thio)methyl]benzamide;
N-(2,4-dimethoxybenzyl)-3-[({2-[(2,2-dimethylpropanoyl)amino]phenyl}thio)methyl]benzamide;
N-{2-[(3-{[(2,4-dimethoxybenzyl)amino]carbonyl}benzyl)thio]phenyl}pyridine-2-carboxamide;
N-{2-[(3-{[(2,6-dimethoxybenzyl)amino]carbonyl}benzyl)thio]phenyl}pyridine-2-carboxamide;
2-({2-[(3-{[(2,4-dimethoxybenzyl)amino]carbonyl}benzyl)thio]phenyl}amino)-2-oxoethylacetate;
3-[({2-[(3-{[(2,4-dimethoxybenzyl)amino]carbonyl}benzyl)thiophenyl}amino)carbonyl]-2-methylphenyl acetate;
2-({2-[(3-{[(2,4dimethoxybenzyl)amino]carbonyl}benzyl)thio]phenyl}amino) -1-methyl-2-oxoethyl acetate;
2-({2-[(3-{[(2,4-dimethoxybenzyl)amino]carbonyl}benzyl)thio]phenyl amino)-2-oxo-1-phenylethyl acetate;
N-{2-[(3-{[(2,4-dimethoxybenzyl)amino]carbonyl}benzyl)thio]phenyl}-2-methoxybenzamide;
N-{2-[(3-{[(2,4-dimethoxybenzyl)amino]carbonyl}benzyl)thio]phenyl}nicotinamide;
N-(2,4-dimethoxybenzyl)-3-{[(2-{[N-(2methoxyethyl)glycyl]amino}phenyl)thio]methyl}benzamide;
N-(2,4-dimethoxybenzyl)-3-[({2-[(piperidin-1-ylacetyl)amino]phenyl}thio) methyl]benzamide;
N-(2,4-dimethoxybenzyl)-3-{[(2-{[N-(tetrahydrofuran-2-ylmethyl)glycyl]amino}phenyl)thio]methyl}benzamide;
N-(2,4-dimethoxybenzyl)-3-[({3-[(2,2-dimethylpropanoyl)amino]pyridin-2-yl}thio)methyl]benzamide;
3-[({2-[(cyclopentylcarbonyl)amino]phenyl}thio)methyl]-N-(2,4-dimethoxybenzyl)benzamide;
N-(2,4-dimethoxybenzyl)-3-{[(2-{[(1-phenylcyclopropyl)carbonyl]amino}phenyl)thio]methyl}benzamide;
3-({[2-({[1-(4-chlorophenyl)cyclopentyl]carbonyl}amino)phenyl]thio}methyl)-N-(2,4-dimethoxybenzyl)benzamide;
6-chloro-N-{2-[(3-{[(2,4-dimethoxybenzyl)amino]carbonyl}benzylthio]phenyl}nicotinamide;
6-chloro-N-{2-[(3-{[(2,6-dimethoxybenzyl)amino]carbonyl}benzyl) thio]phenyl}nicotinamide;
3-({2-[(3-chloro-2,2-dimethylpropanoyl)amino]benzyl}thio)-N-(2,4-dimethoxybenzyl)benzamide;
3-({2-[(cyclopentylcarbonyl)amino]benzyl}thio)-N-(2,4-dimethoxy benzyl)benzamide;
N-(2,4-dimethoxybenzyl)-3-({2-[(2,2-dimethylpropanoyl)amino]benzyl}thio)benzamide;
3-({2-[(3-chloro-2,2-dimethylpropanoyl)amino]benzyl}thio)-N-(2,6-dimethoxybenzyl)benzamide;
3-({2-[(cyclopentylcarbonyl)amino]benzyl}thio)-N-(2,6-dimethoxybenzyl) benzamide;
N-(2,6-dimethoxybenzyl)-3-({2-[(2,2-dimethylpropanoyl)amino]benzyl}thio)benzamide;
N-(2,6-dimethoxybenzyl)-3-({2-[(trichloroacetyl)amino]benzyl}thio) benzamide;
N-(2,6-dimethoxybenzyl)-3-({2-[(3,3-dimethylbutanoyl)amino]benzyl}thio) benzamide.

28. A process for the treatment or prevention of a condition in a mammal which is modulated by LXR, comprising administering to a mammal in need thereof a therapeutically effective dose of a compound according to claim 1.

29. A process for the treatment or prevention of a condition in a mammal which is modulated by LXR, comprising administering to a mammal in need thereof a therapeutically effective dose of a compound according to claim 9.

30. A process for the treatment or prevention of a condition in a mammal which is modulated by LXR, comprising administering to a mammal in need thereof a therapeutically effective dose of a compound according to claim 27.
